(19) United States
(12) Patent Application Publication
(10) Pub. No.: US 2012/0283601 A1
(43) Pub. Date: Nov. 8, 2012

(54) Method and Device for Restoring Spinal Disk Functions

(76) Inventor: Wolfgang R. Daum, Groton, MA (US)

(21) Appl. No.: 13/464,859

(22) Filed: May 4, 2012

Related U.S. Application Data
(63) Continuation-in-part of application No. 12/070,161, filed on Feb. 15, 2008.
(60) Provisional application No. 60/903,441, filed on Feb. 26, 2007.
(60) Provisional application No. 60/918,366, filed on Mar. 16, 2007.
(60) Provisional application No. 60/922,707, filed on Apr. 10, 2007.
(60) Provisional application No. 60/923,653, filed on Apr. 16, 2007.
(60) Provisional application No. 60/993,107, filed on Sep. 10, 2007.

(51) Int. Cl. A61B 5/103; A61B 17/58; A61B 17/16

(52) U.S. Cl. 600/594; 606/80; 606/93

(57) ABSTRACT

The present invention relates to medical devices, systems and methods for restoring spinal disc function. The invention provides a curved path through the vertebra into the disc through which the disc can be filled with an augmenting substance, balloon, or pellets. Further, the delivery device can be used to access the disc with surgical instrumentation. The invention further discloses a responsive disc augmentation system.

### CROSS REFERENCE TO RELATED APPLICATIONS

This application is a continuation-in-part of, and claims the benefit of and priority to U.S. application Ser. No. 12/070,161, currently pending, which takes priority to provisional patent application Ser. No. 60/903,441 filed on Feb. 26, 2007; provisional application Ser. No. 60/918,366 filed on Mar. 16, 2007; provisional application Ser. No. 60/922,707 filed on Apr. 10, 2007; provisional application Ser. No. 60/923,653 filed on Apr. 16, 2007; and provisional application Ser. No. 60/993,107 filed on Sep. 10, 2007; all of which are incorporated herein by reference.

TECHNICAL FIELD

The present invention relates to an improved system of methods and devices for restoring spinal disc function. The invention provides a curved path through the vertebra into the disc as a delivery system through which therapy can be provided for the disc. The invention further provides various methods and devices to treat the disc, such as gels and liquids, for an augmentation of the disc or as radiographic contrast media; or use the use of balloons, pellets, and the like as part of treatment.

### BACKGROUND

A common disorder of the lower spine is disc degeneration, also called degenerative disc disease (DDD) or osteoarthritis in the spine. The human vertebral column (spine) is made from bony vertebrae separated by soft tissue inter-vertebral discs. These spinal discs are flexible joints which provide for flexion, extension, and rotation of the vertebrae relative to one another, and therefore contributing to the stability and mobility of the spine within the axial skeleton. Each of these spinal discs consists of an outer annulus fibrosus, which surrounds the inner nucleus pulposus. The annulus fibrosus consists of several layers of fibrous cartilage. The strong annular fibers contain the nucleus pulposus and distribute pressure evenly across the disc. The nucleus pulposus contains loose fibers suspended in a mucoprotein gel, having a gelatinous consistency. The nucleus pulposus plays a central role in maintaining normal disc function. The nucleus of the disc acts as a shock absorber, absorbing the impact of the body's activities and keeping the two vertebrae separated. If one presses down on the front of the disc the jelly moves posteriorly or to the back. When one develops a prolapsed disc, the jelly/nucleus pulposus is forced out of the disc and may put pressure on nerves located near the disc. This creates the symptoms of sciatica.

As people age, the nucleus pulposus begins to dehydrate, which limits its ability to absorb shock. The annulus fibrosus gets weaker with age and begins to tear. One generally refers to the gradual dehydration of the nucleus pulposus as degenerative disc disease or DDD, which is most common in the lower spine. DDD is actually not a disease but, rather, a degenerative condition that can be painful and can greatly affect the victim's quality of life.

Often, degenerative disc disease can be successfully treated without surgery. Physical therapy, anti-inflammatory medications such as non steroidal anti-inflammatory drugs, chiropractic treatments, or spinal injections often provide adequate relief of these troubling symptoms. Surgery may be recommended if the conservative treatment options do not provide relief within 2 to 3 months. If leg or back pain limits normal activity, if there is weakness or numbness in the legs, if it is difficult to walk or stand, or if medication or physical therapy are ineffective, surgery may be necessary, most often spinal fusion. Bone grafts or artificial disc replacement may be an option in treating DDD under certain conditions. Various implants have been designed to overcome DDD. However, all these devices and treatments are very invasive.

Some approaches are known to restore the normal self-sustaining hydrodynamic function of the disc by injecting various substances. These include hydrogels; biodegradable polymers; microparticulates and collagen which can be cross-linked by exposure to ultraviolet radiation; bioactive glass; polymer foam and polymer foam coated with sol gel bioactive material; small particles or other fatty tissue for use as a carrier, and adding to the carrier growth factors such as transforming growth factor beta (TGF-.beta.) and bone morphogenic protein (BMP); or cross-linkable compositions in which the viscosity may be controlled. Early work on injectable disc augmentation includes Smith U.S. Pat. No. 3,320,131.

One difficulty in injecting these disc augmenting substances directly into the disc is that after needle removal the fluid will leak out again through the channel which was formed by the injecting instrument. It has therefore been proposed to enter the disc through the vertebral bone, since the bone can easily be closed with bone cement. Current methods have in common that they inject the gel with a conventional needle type instrument (syringe) into the disc, cutting through the annulus fibrosus. However, in contrast to blood vessel walls, the disc wall—the annulus fibrosus—does not have the means to heal itself. Thus, an incision through the annulus fibrosus leaves a permanent perforation. This leads to a leak through which the gel will escape and also builds a starting point for further rupture of the annulus fibrosus as the patient continues moving the spine. Natarajan et. al. demonstrated these negative effects of direct incision in his paper “Effect of annular incision type on the change in biomechanical properties in a herniated lumbar intervertebral discs”. J Biomech Eng. 2002 April; 124 (2):229-36.

One improvement in approach is to inject the gel through the vertebra (trans-endplate), as described in U.S. 2004/0228853 and in related filings of these inventors; or in Johannessen et. al., Annals of Biom. Eng. 34 (4):687-96, 2006 April; or in Nakai et. al. “Anterior transvertebral herniotomy for cervical disc herniation”, J Spinal Disord, 2000 February; 13 (1):16-21.

U.S. 2007/0003525 discloses (FIG. 10, paragraph 112-114) delivering a liquid composition via 18-31 gauge straight needles and pressure-mediated syringe through the pedicle of the vertebra bone into the nucleus pulposus. In particular, the cross-linked matrix can be administered percutaneously via a biopsy cannula inserted through a canal in the pedicle. After delivery of the matrix component, the canal can then be filled with bone cement or other like material to seal the canal. The device as used in this method is a straight cannula or biopsy instrument. However, due to the straight nature of these instruments and due to the complex human anatomy of the spine it is difficult to direct the instrument through the vertebra bone into the nucleus pulposus or any part of the disc.

Gragg discloses in U.S. 2002/0173796 a trans-sacral axial and trans-vertebral axial method and device to augment a spinal disc. Referring to FIGS. 12 and 13 of '796, the device is forming a channel 152 through the vertebral bodies from an exterior position into a disc nucleus pulposus for injecting an expandable balloon or sack or other envelope or injecting a medium. Finally the cavity is sealed with bone cement. To use this disclosed mechanism to augment a damaged disc is very complicated to implement and requires a rather invasive procedure through the sacrum and various vertebrae to finally reach the targeted disc.

Myint discloses in U.S. 2006/0253198 (FIGS. 13 and 14) a multi-lumen system in which one lumen enters the disc through the vertebra and the other through the annulus. Thus the system does not leave the annulus intact but ruptures it.

U.S. Pat. No. 6,949,101 discloses a medical instrument for milling a curved path in bone and procedure. However, this mechanism is very complex, and it is correspondingly difficult, if at all possible, to drill small diameter curved holes into vertebra bone by this method.

U.S. Pat. No. 6,572,593 discloses a general deflectable needle assembly, which includes a telescoping cannula made from an elastic material. However, said instrument is made from elastic materials such as nickel titanium NiTi, and hence is not clearly strong enough to penetrate bone. Further, the tip of the needle is beveled only to one side, which is fixed in relative orientation, and a drilling procedure as proposed in the present invention is not useable.

U.S. Pat. No. 7,776,068 to Ainsworth et al describes a technique for immobilizing or repairing vertebrae in the lumbar region via a trans-sacral approach with an instrument travelling upward through the spine,

US application 2005/0261684 to Shaolian et al. describes expansion of spinal discs by the insertion of objects which can increase the intervertebral spacing. This approach can help, but the formation of a fixed pivot within a disc does not repair the natural flow of the intradisc fluids within the disc, not does it provide a shock-absorbing function.

### SUMMARY OF THE INVENTION

It is the object of the present invention to provide improved minimally invasive methods and devices for administering improved disc augmenting compositions. In one aspect, the goal of the present invention is to provide a device, system and method to access the spinal disc percutaneously by drilling an access path through the pedicle and vertebra body. Due to the vertebral anatomy, it is necessary to have the injectable instrument follow a curved path within the vertebral bone. To provide an instrument capable of performing a curve in a bone structure is one of the goals of this invention. Through the channel obtained by this route, the disc can be reached with various treatment means. After deploying the treatment means or finishing the disc procedure, the curved access channel is sealed with conventional bone cement. The advantage of this technique or of a delivery system utilizing this technique is, that it leaves the annulus fibrosus of the disc intact (after permanently sealing an opening through the vertebral bone by use of bone cement), and also, as a percutaneous and minimally invasive procedure, leaves other outer parts of the vertebra, pedicle and disc untouched, thereby allowing for further treatments.

One embodiment of the present invention is a treatment device, system and method by which a floatable or liquid spinal support medium is injected via the delivery system into the mucoprotein gel of the nucleus pulposus. The difficulty with current techniques which inject the floatable solution into the disc through the annulus fibrosus is that after the needle is removed, the fluid, which in its simplest form can be water, will leak out again through the hole which was formed by the injecting needle.

Another embodiment of the present invention is a treatment device, system and method by which one or a number of delivery systems are permanently left in various discs and connected to a central unit capable of pumping further gel into the disc. As the disc ages, various disease or age originated leaks of the annulus fibrosus cause constant shrinkage or out-diffusion of the mucoprotein gel of the nucleus pulposus. The system as invented here can adjust for this chronic shrinkage by steady or user controlled pumping of disc augmenting gel into the nucleus disc space.

Another embodiment of the present invention is a treatment device, system and method by which a balloon filled with a gel or liquid is deployed and anchored via the delivery system into the nucleus of the disc. The balloon or the balloons comprise a string which is left in the sealing bone cement of the curved channel, which anchors them and hold them in a defined position in the disc. Previous attempts to augment the disc with balloons had the difficulty that the balloons migrated within the disc, especially towards the back of the disc, and hindered correct spinal movement.

Another embodiment of the present invention is a treatment device, system and method by which pellets made from a rubbery material are deployed into the disc via the delivery system. Just like the balloons as described above, these pellets can be anchored within the disc.

The above summary of the present invention is not intended to describe each illustrated embodiment or every implementation of the present invention. The figures and the detailed description which follow particularly exemplify these embodiments.

### BRIEF DESCRIPTION OF THE DRAWINGS

FIG. 1 (1a-1b) illustrates the path through the vertebra: a) horizontal or cranial view, b) left lateral or sagittal view.

FIG. 2 illustrates a cross sectional view of the spine with 2 vertebra and 3 discs and a delivery system to inject gel into the disc: a) Percutaneous access of the pedicle; b) Straight drilling through the pedicle into the vertebra body; c) After removal of straight drill; d) Curved drilling upwards to reach the disc (here not in the center of the disc); e) After removal of the curved drill to inject a gel into the disc; f) Injection of bone cement to close and seal the disc access channel; g) After removal of the straight percutaneous puncture needle.

FIG. 3 illustrates the procedural sequence method of using the delivery system to inject gel into the disc: a) Anchor is placed onto the pedicle, b) Attaching motor unit and drilling through the pedicle into the vertebra, c) Drilling curved canal, d) After removing motor unit and leaving a canal, e) Injecting disc augmenting substance, f) Injecting bone cement, g) After removing all instruments.

FIG. 4 illustrates the principal system of the spinal disc augmentation system.

FIG. 5 illustrates a cross sectional view of one spinal vertebra with two adjacent discs: a) Straight inserted instrument (stiff tube), b) Curvature of the elastic and drilling tube within the vertebra towards the superior disc, c) Drilled cavity or canal in vertebra, d) Tube inserted through canal in vertebra to disc.

FIG. 6 illustrates a cross sectional view of one spinal vertebra with two adjacent discs: a) Straight inserted instrument (stiff tube); b) Curvature of the elastic and drilling tube within the vertebrae towards the superior disc; c) Drilled cavity or canal in vertebrae; d) Tube inserted through canal in vertebrae to disc; e) Balloon inserted in which a substance is injected; f) Balloon closed and bone cement injected in canal to seal canal.

FIG. 7 illustrates a cross sectional view of one spinal vertebra with two adjacent discs.

FIG. 8 illustrates a cross sectional view of one spinal vertebra with two adjacent discs: a) straight inserted instrument (stiff tube); b) curvature of the elastic and drilling tube within the vertebra towards the superior disc; c) drilled cavity or canal in vertebra; d) pushing of tablets or pellets and bone cement through the canal into the disc; e) all tablets have been pushed into the disc; f) all delivery instruments removed.

It should be noted that sizes, dimensions or measurements in the figures do not reflect actual device or anatomical geometry, but are for illustrational purpose of the device and method principal only.

### DETAILED DESCRIPTION OF EXEMPLARY EMBODIMENTS

### 1. Definitions

The terms “fluid”, “floatable”, “liquid”, “matrix”, “component”, “agent”, “jelly”, “gel”, “substance”, “solution” as used herein, refer to a substance having a viscosity which allows the substance to be injected into a spinal disc via the device, system or method of the present invention. The present invention makes use of various terms to reflect the various aspects of the substance and to refer if needed to the description of referred art in the field.

The term “distal” as used herein refers to a location farther away from the performing physician, usually further in the patient's body. The term “proximal” as used herein, refers to a location closer to the performing physician. The term “path” as used herein refers to the trajectory or route of a penetrating medical instrument. A path can be a channel or canal. The term “bevel” as used herein, refers to an angle other than a right angle of the tip of a needle or a hallow tube. To bevel usually means transforming a tube into a needle. A needle can be beveled to just one side giving the tube tip a sharp edge on just one side of the outer tube surface, or a needle is beveled circular giving a circular sharp edge at the inner tube surface.

The term “torque wire” as used herein, refers to a wire which when rotated around its axis on the proximal side results in the same rotation on its distal part. The term “elastic” as used herein, refers to a reversible deformation of the pre-bend tube of the device, while a plastic deformation is irreversible and does not bend back to its original shape.

The term “less elastic” as used herein, refers stiff or rigid tube capable of restraining a pre-bend elastic tube to a straight one. Stiffness is the resistance of an elastic body to deflection or deformation by an applied force. The term “pre-bend” or “pre-curved” refers to a straight elastic tube which is bent to a non-reversible non-straight shape, such as a curved shape. The term “vertebrae” (singular: vertebra) refers to the individual irregular bones that make up the vertebral column or spine of the human and include and include the cervical, thoracic and lumbar bones, the five bones that are fused to form the sacrum, and the four which form the tailbone.

The term “tablet” as used herein refers to a small and rounded mechanical device, pill, capsule, pellet or caplet made from an elastic or rubber type material. A tablet can be in its simplest form a rounded piece of silicon rubber, like a rubber ball. A tablet can also be a small plastic, metal or rubber capsule or rubber balloon type device filled with a substance like a hydrogel or with a colloidal suspension.

### 2. Principle of the Invention

The present invention proposes to reach the disc from the patient's skin via a curved path through the vertebra bone, said path being suitable for the filling of a spinal disc with an augmenting liquid substance. The present invention proposes to puncture a needle assembly type device percutaneously through the patient's skin and underlining soft tissue, and into the pedicle of a vertebra, drilling through the pedicle and vertebra bone in a curved path to reach the disc and provide a canal, injecting the augmenting substance into the disc, and closing or sealing the vertebra bone with bone cement.

Ideally the disc is punctured approximately perpendicularly along the local axis of the spine. For drilling a curved canal through the vertebra bone, the present invention proposes a “pre-bend” elastic tube, i.e., a tube having a pre-formed bias to form a bend at a location on the elastic tube. The pre-bend tube may have a drill at its distal tip. The pre-bend tube is initially restrained to a straight shape by a straight rigid tube, which is less elastic than the pre-bend tube. When pushed away from the restraining rigid tube and into the vertebra while rotating the drill, the superposition of forward force and sideward force—due to the tendency of the elastic tube to return to its original bend shape—results in a curved path through the vertebra. In the present embodiment of the system, the pre-bend elastic tube is located around the restraining less elastic tube. In one aspect, the invention provides means to move a pre-bend elastic tube away from its restraint by a less elastic tube while using a drill mechanism at the tip of the elastic tube to drill though bone matter.

In a preferred embodiment, the curved path can reach from the backside of the vertebra towards the front (anterior) side, then through the pedicle of the vertebra and up into the vertebra (superior) or down into the vertebra (inferior). In some cases the curved path can reach from the front side of the vertebra towards the back (posterior) up into the vertebra (superior) or down into the vertebra (inferior).

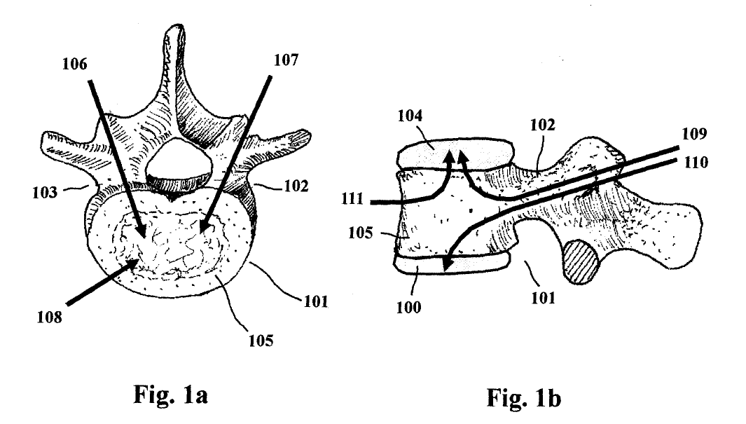

FIG. 1 illustrates, in a view along the spine (FIG. 1a) and a cross-section of a vertebra (FIG. 1b), several possible pathways through the pedicles 102 and 103 of the vertebra 101 into the discs 100 and 104. Arrow 106 indicates a path from the right pedicle 103 through the corpus vertebra 105. Arrow 107 indicates a path through the left pedicle 102 through the corpus vertebra 105. In one embodiment of the present invention the path as indicated by arrow 111 comes from the front side of the patient's body in dorsal direction directly into the corpus vertebra 105. Arrow 109 indicates a path through the pedicle 102, curving in the corpus vertebra 105 in superior (upper) direction to disc 104. Arrow 110 indicates a path through the pedicle 102, curving in the corpus vertebra 105 in inferior (lower) direction to disc 100. Arrow 111 indicates a path directly through the corpus vertebra 105 in superior direction to disc 104.

### 3. Example 1

### Acute Disc Gel Augmentation

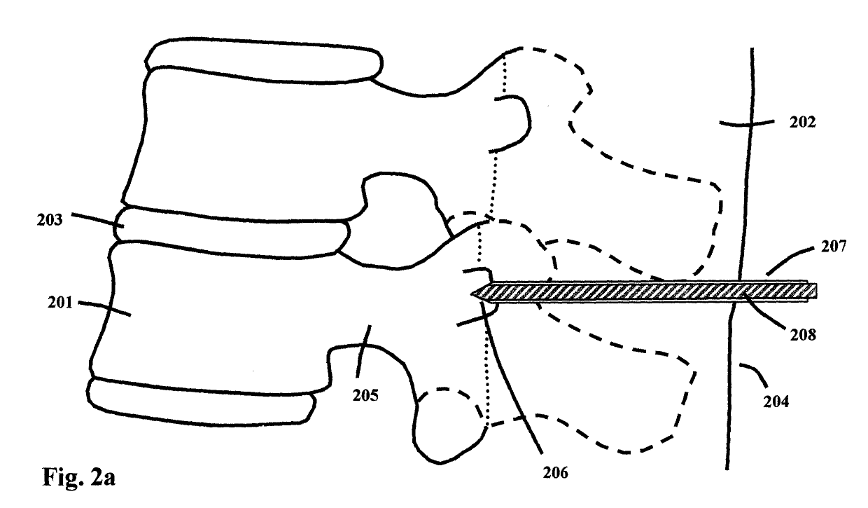

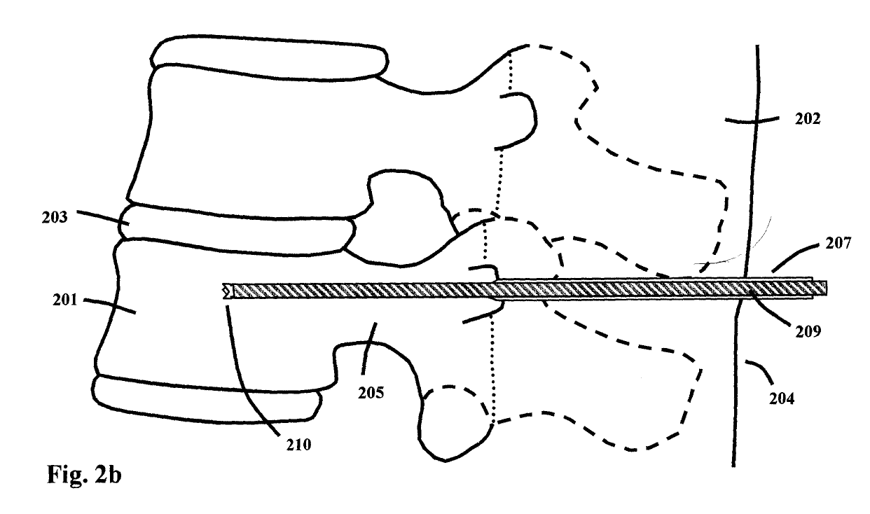

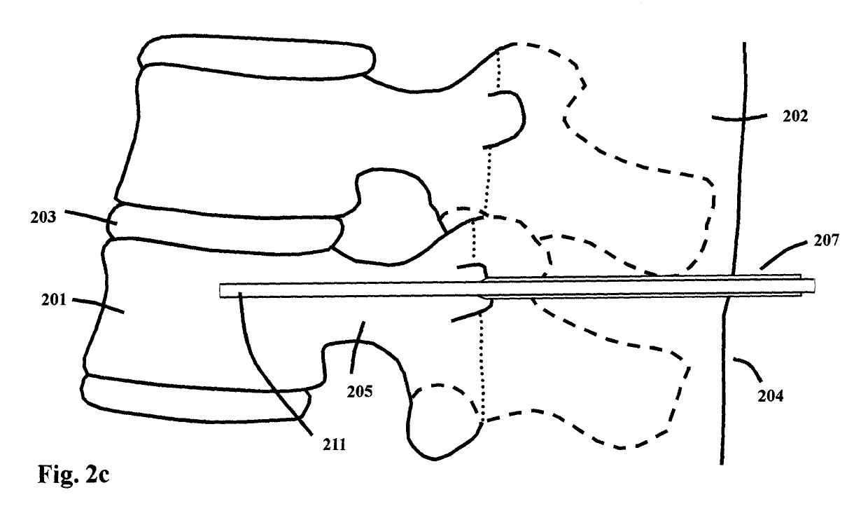

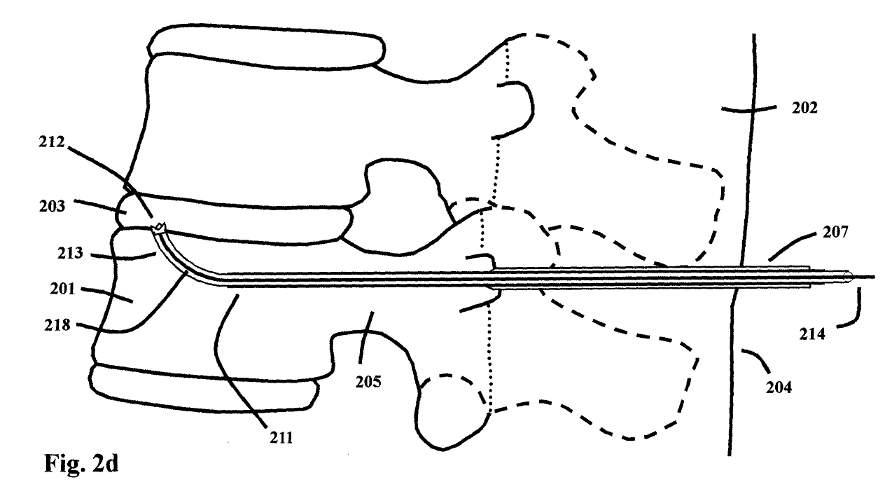

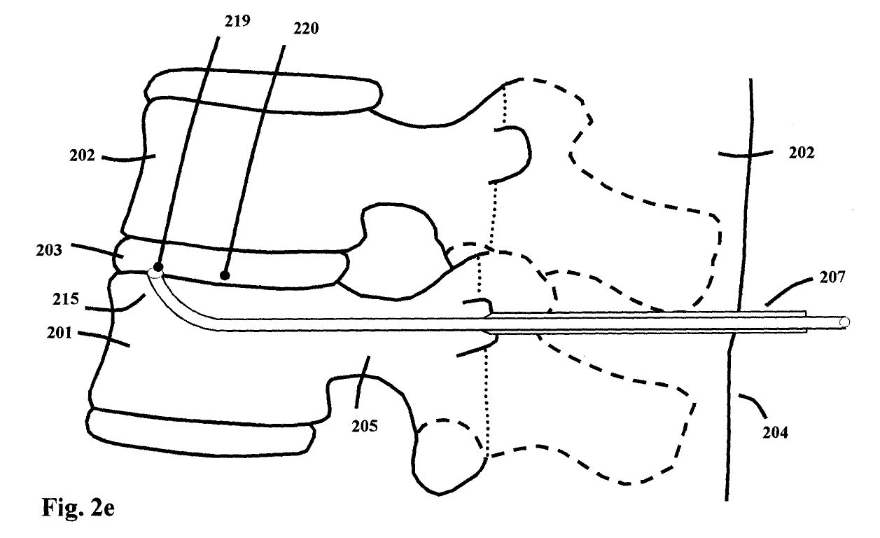

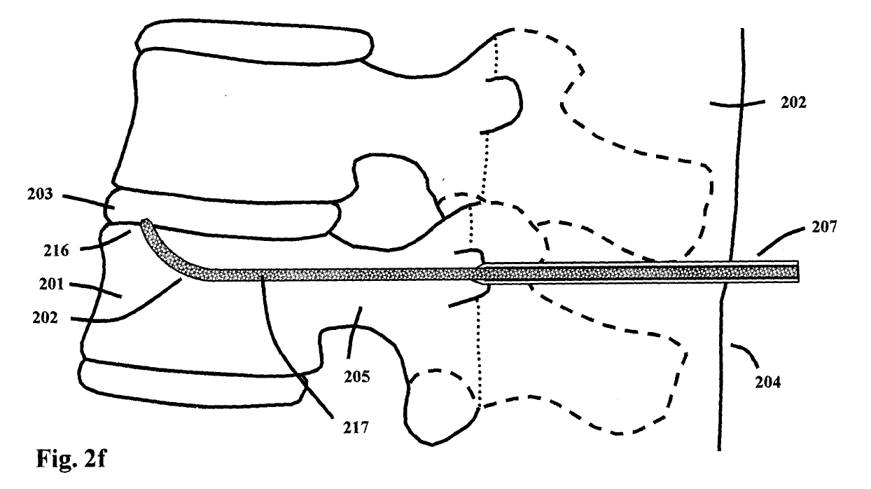

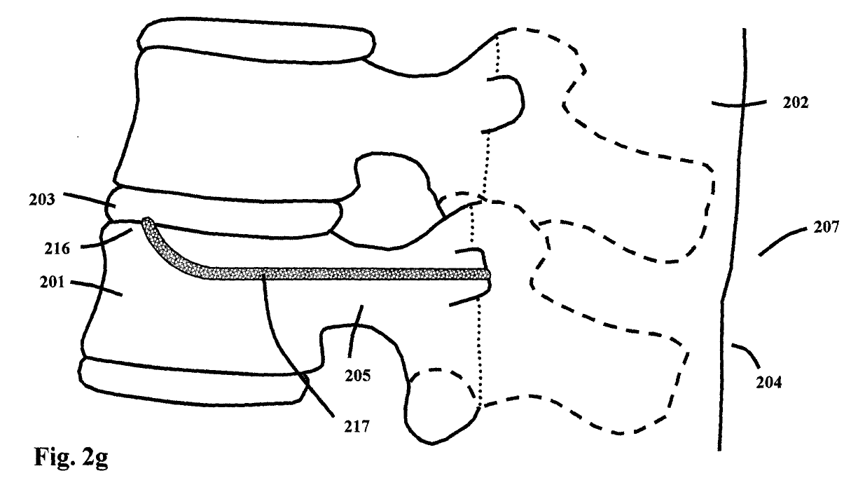

FIG. 2 illustrates one embodiment of the present invention in which the disc 203 is accessed through the lower adjacent vertebra 201.

FIG. 2a. illustrates the step in which the instrument has been pushed—e.g. by hand force—through the patient's skin 204 and underlying soft tissue 202 onto the bone surface of the pedicle 205. The instrument comprises a stiff and straight outer tube 207, or anchor tube, and an inner obdurator 208 beveled to build a sharp front edge 206, which allows the instrument to penetrate through the skin 204 and tissue 202.

FIG. 2b illustrates the next step. After the obdurator 208 was proximally removed, a drill rod 209 with drill 210 at its distal end was inserted in the inner lumen of straight outer guiding tube 207 to drill a straight path through the pedicle 205 into the vertebra body 201.

FIG. 2c illustrates the step in which all drilling parts have been pulled back and removed and a straight guiding tube 211 was inserted through the straight outer tube 207 into the vertebra body 201.

FIG. 2d illustrates the step in which a curved path is drilled through the vertebra body 201 into the disc 203. To do this a drill 212, which can be designed as a conventional bone drill or swivel, is connected to a torque wire 214, which rotates the drill 212. The torque wire 214, preferably made from metal, is held in the center of the pre-bent elastic tube 213 by an elastic plastic catheter 218 shown in white in the figure. The drill 212 is mounted on the distal tip of a pre-bent elastic tube 213. In the initial stage of the drilling, the pre-bent tube 213 is fully inserted and restrained in the straight and stiff guiding tube 211. The pre-bent tube 213 has due to it's pre-bent nature the tendency to bend back to it's original pre-bent curved form when pushed outwards and release from tube 211 in distal direction. Constantly rotating the drill 212 via torque wire 214 from a motor (not shown in this figure), while pushing forward the assembly of drill 212 and pre-bent tube 213 in a distal direction, allows the assembly to drive (drill) a curved bore hole through the vertebra bone towards the disc 203. It is the superposition of forward speed of the assembly and tendency of the pre-bent tube 213 to bend back to its original bent shape or form which results in the curved hole. Thus rotation speed of the drilling and forward speed of the assembly need to be matched carefully. Further, for this to work the guiding tube 211 needs to be designed to fully restrain the pre-bent tube 213 into a straight shape. The pre-bent tube 213 can be made from a super-elastic material like nickel-titanium as described further below. Not shown in the figure is a suction mechanism drawing the bone shavings out to the proximal side of the device. Further, there can be a rinsing mechanism rinsing the bone shavings from the tube.

FIG. 2e illustrates the situation in which the drilling assembly is fully pulled back and removed and an access path to the disc 203 remains through the vertebra body 201 and pedicle 205 and straight stiff tube 207. In this illustrative example the access point 219 through the endplate of the disc/vertebra is located more to the front side of the disc 203. Ideally one would try to access the disc 203 further at the middle or center point 220. Through this channel or canal 215 disc augmenting substance or gel can be injected.

FIG. 2f illustrates the situation in which the disc 203 was injected with a disc augmenting substance and the canal 215 was sealed with bone cement 217. In order to avoid injecting the bone cement beyond the disc entry opening 216 into the disc 203, the bone cement preferably comprises radiographic contrast media, and the procedure is performed under radiographic imaging, which can be X-ray, computer tomography, magnetic resonance imaging, ultrasound, PET or SPECT or any other radiographic method.

FIG. 2g illustrates the situation in which all instrumentation has been removed from the patient's body and only the bone cement 217 is left in the vertebra 201 to seal the opening 216 of the disc 203 and to fill the passage created in the procerure.

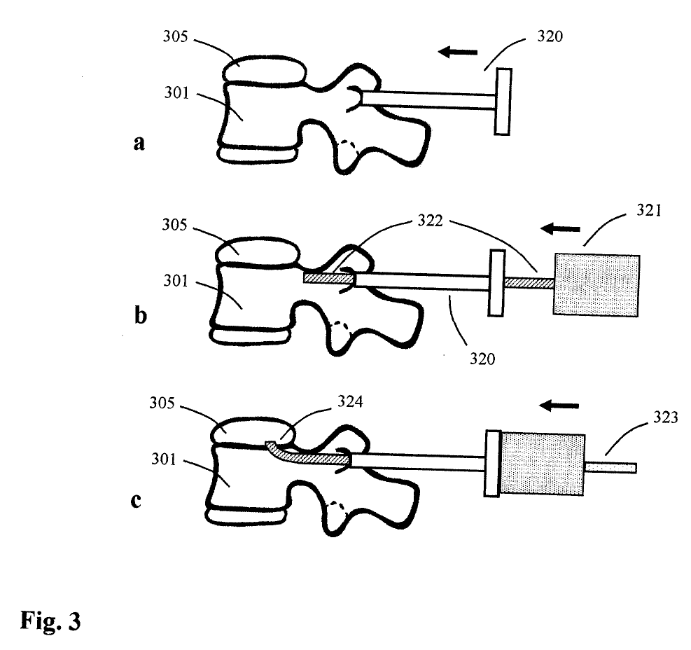

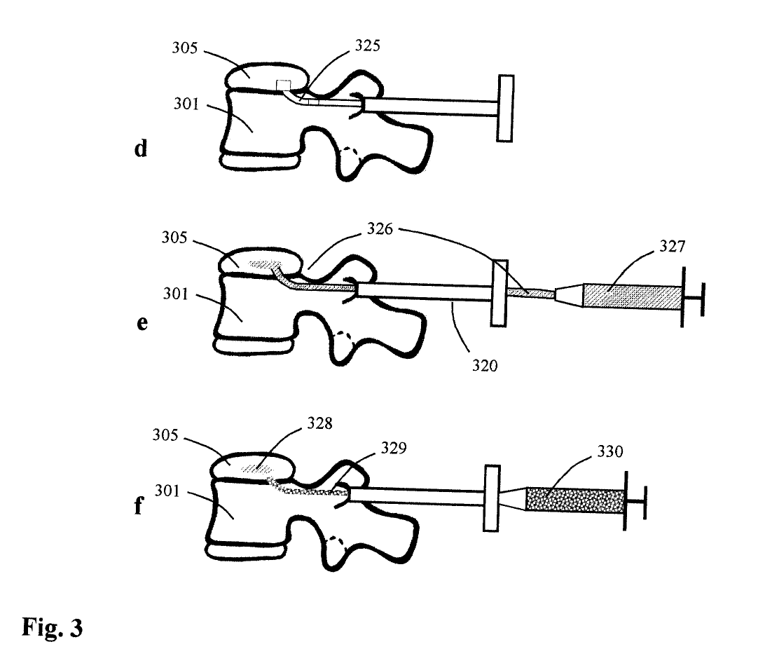

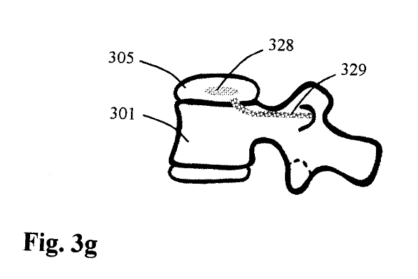

FIG. 3 schematically illustrates procedural aspects of the method to augment the disc 305 through a curved access in the vertebra 301.

The injection unit to inject the disc augmenting substance is in its simplest form a hand held syringe but can also a motor powered injector system allowing the injection pressure to be automatically controlled. The injection unit to inject the bone cement is in its simplest form a hand held syringe but can also a motor powered injector system allowing the injection pressure to be automatically controlled.

The injectable substance can be a liquid, juice, paste, jell, gel, powder, coagulate, or a particle conglomerate. Some disc augmenting substances or bone cements may need special treatment, such as light illumination for cross linking or hardening of the substance. The system may comprise special means to insert utilities which are needed for said special treatment. Such a means could be an opening to insert a glass-fiber to illuminate the substance. The appropriate light source could be part of the system.

The injectable substance typically has the purpose of mechanically stabilizing the disc and thus the spine. However, the injectable substance may in addition have a therapeutic effect on the spinal disc itself, for example for radiographic contrast imaging, or for radiographic enhancement imaging, or for radiographic marking, or for a preventive or anti-aging purpose.

Any injectable bone cement can be used, such as polymethylmethacrylate (PMMA) or hydroxyapatite. Many such cements are hardenable by polymerization, cross linking, ionic bonding, or other conventional means.

In its simplest form the disc augmenting agent is water or an isotonic solution. But many injectable disc augmenting substances exist which can be used. The viscosity of the disc augmenting substances may vary from 1 centiPoise to 1,000,000 centiPoise.

In FIG. 3a, the stiff anchor tube 320 is pushed in the direction of the arrow, and anchored onto the targeted vertebra 301. In FIG. 3b, the pre-bend straightened elastic tube 322, on the tip of which the drill (not shown) is rotating, is adapted to be pushed (arrow) on its proximal side by the motor unit 321 through the stiff anchor tube 320. In FIG. 3c, the motor unit 321 is further pushed forward distally while the stiff inner tube 323 is kept still or unmoved. This causes the bending 324 of the elastic tube 322 towards the superior disc 305 while it is being pushed forward distally. Notice that order to show variations of the method in this case, in contrast to the method shown in FIG. 2, the disc 305 is accessed further to the back of the disc. FIG. 3d illustrates the empty canal 325 within the vertebra 301 after the elastic tube and motor unit have been pulled back and removed. To demonstrate that there can be variations to the method, in contrast to the method as illustrated in FIG. 2 where the disc augmenting gel was injected right through the bone cavity, FIG. 3e illustrates a step in which an elastic catheter 326 is inserted through the anchor tube 320 into the disc 305 and disc augmenting substance 328 (see FIG. 3f) is injected from a hand operated syringe 327. During this step other instruments, such as a glass fiber emitting light (not shown), can be inserted through the catheter 326 to treat the disc augmenting substance. FIG. 3f illustrates the situation in which the tube 326 in removed and bone cement 329 is injected from a second hand operated syringe 330 into the canal 325 in the vertebra 301 to close and seal the canal 325. Finally FIG. 3g illustrates the situation in which all instruments are removed and the disc 305 is augmented with disc augmenting substance 328 and the canal 325 in the vertebra 301 is sealed with bone cement 329.

### 4. Example 2

### Chronic Disc Gel Augmentation

One embodiment of the invention is a responsive disc augmentation system, which measures the pressure in the spinal disc and releases disc augmenting substance into the disc when the pressure drops under a critical value. The system can be set for autonomous or manual augmentation release. The pressure is typically measured when the patient is prone or supine, for instance at night.

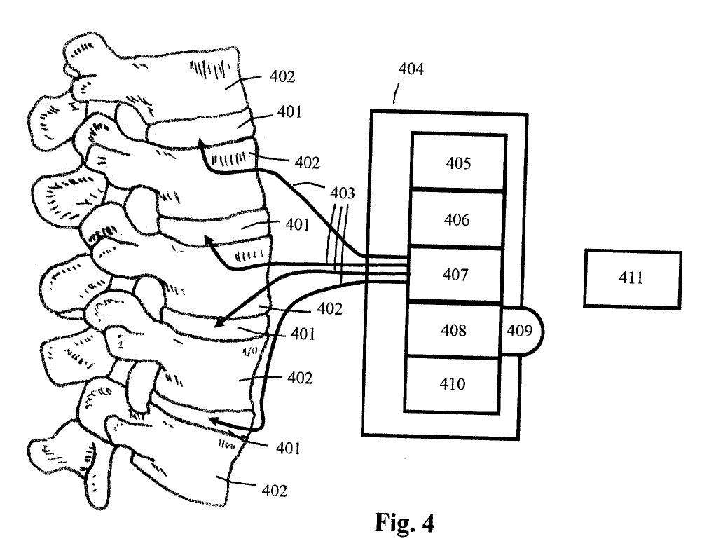

FIG. 4 illustrates the schematic principle of the fully implantable system. The implantable can or canister 404 contains the control unit 405, radio frequency communication unit 406, pump unit 407 for the augmenting substance, augmenting substance reservoir 408 with refill subunit 409, and power unit 410. Implantable tubing connects at least one of the inner lumen of the spinal discs 401 with the pump unit 407. The discs 401 can be reached by direct puncture through the annulus fibrosus of each disc or via the endplates of the adjacent vertebrae 402. A non-implanted external communication console 411 allows the changing of procedural settings of the system, and read out of parameters, for example the filling of the reservoir 408.

The canister 404 is biocompatible and can be implanted anywhere within the body, preferably subcutaneously close to the area of the augmented spinal discs. Because the most serious disc degeneration takes place in the lumbar or cervical spine, the canister 404 is most likely to be implanted subcutaneously in the lumbar region, or else underneath the collarbone. If the system only augments one disc, the canister will be located as close to the disc as possible.

The communication unit 406 communicates via radio frequency with the external console and exchanges data like the amount of energy remaining in the power unit 410, the amount of augmenting substance remaining in the reservoir 408, the amount of augmenting substance injected into each augmented disc 401, and the dates of injection. Further may it be important to read out a complete pressure time measurement for each measured disc 401.

The pump unit 407 for pumping the augmenting substance may comprise a single pump and as many valves as needed for steering the augmenting substance in each augmented disc 401; or alternatively, may comprise as many pumps as needed for steering the augmenting substance in each augmented disc 401. Rotor-dynamic pumps, positive displacement pumps, or any other state of the art implantable drug pumps can be used. A positive displacement pump causes a liquid to move by trapping a fixed amount of fluid and then forcing (displacing) that trapped volume into the discharge pipe. Positive displacement pumps can be further classified as either rotary-type (for example the rotary vane pump) or reciprocating-type (for example the diaphragm pump). Centrifugal Pumps convert the mechanical energy into hydraulic energy by centrifugal force on the liquid. Hydraulic energy is in the form of pressure energy.

The augmenting substance reservoir 408 is basically a tank for the disc augmenting substance to be pumped into the disc 401. The refill subunit 409 is in its most simple form a conventional medical port in which one can inject a substance through a rubber cap. The power unit 410 contains a battery which can either be inductively recharged or explanted and removed with a newly charged one.

At the distal tip of each disc entering tube 403 a pressure sensor will sense the intervertebral disc pressure. The signal is transferred for further processing to the control unit 405 via cable.

To enter the disc posteriorly, the present invention proposes drilling through the vertebra bone in a curved path. For drilling a curved canal through the vertebra bone, the present invention proposes a pre-bend elastic tube with a drill at its distal tip and which is initially restrained to a straight shape by a straight rigid tube. When pushed away from the restraining and less elastic tube into the vertebra while rotating the drill, the superposition of forward force and sideward force—due to the nature of the elastic tube to get into its original shape—results in a curved path through the vertebra.

The pre-bend elastic tube can be located within or around the restraining less elastic tube. The essence of the invention is to move a pre-bend elastic tube away from its restraining or bending less elastic tube while using a drill mechanism at the tip of the elastic tube to drill through bone matter.

The curved path can reach from the backside of the vertebra towards the front (anterior) through the pedicle of the vertebra up the vertebra (superior) or down the vertebra (inferior). In some cases the curved path can reach from the front side of the vertebra towards the back (posterior) up the vertebra (superior) or down the vertebra (inferior).

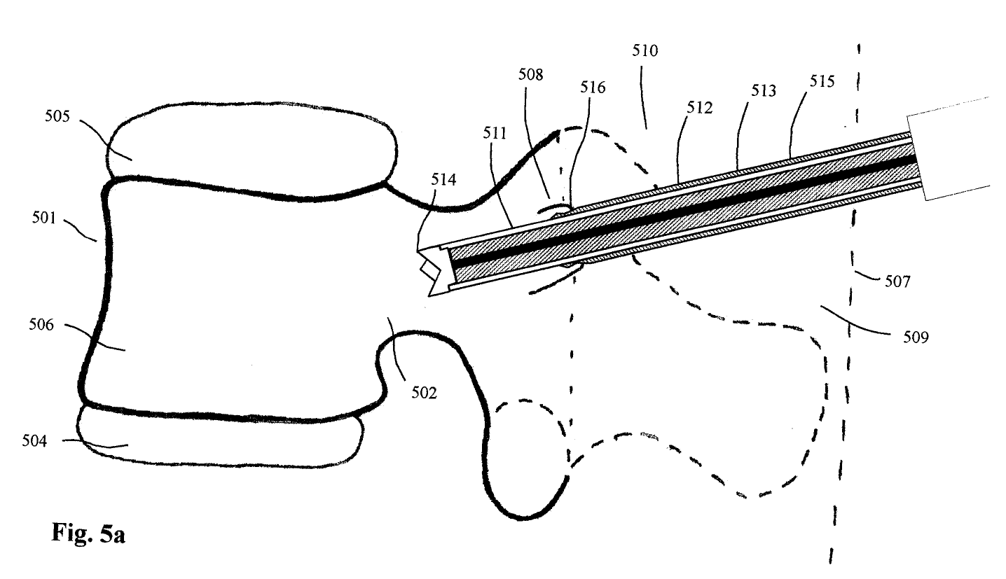

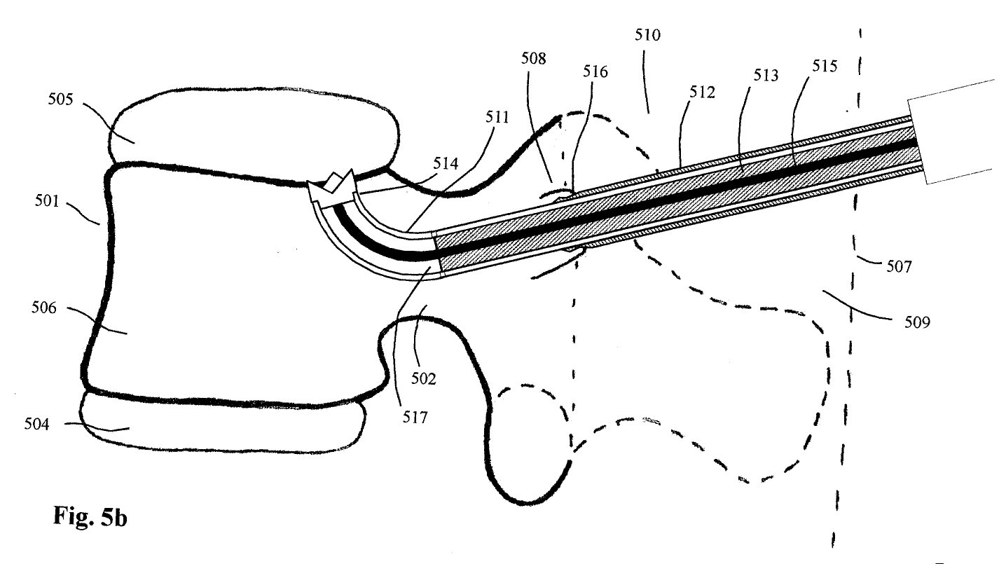

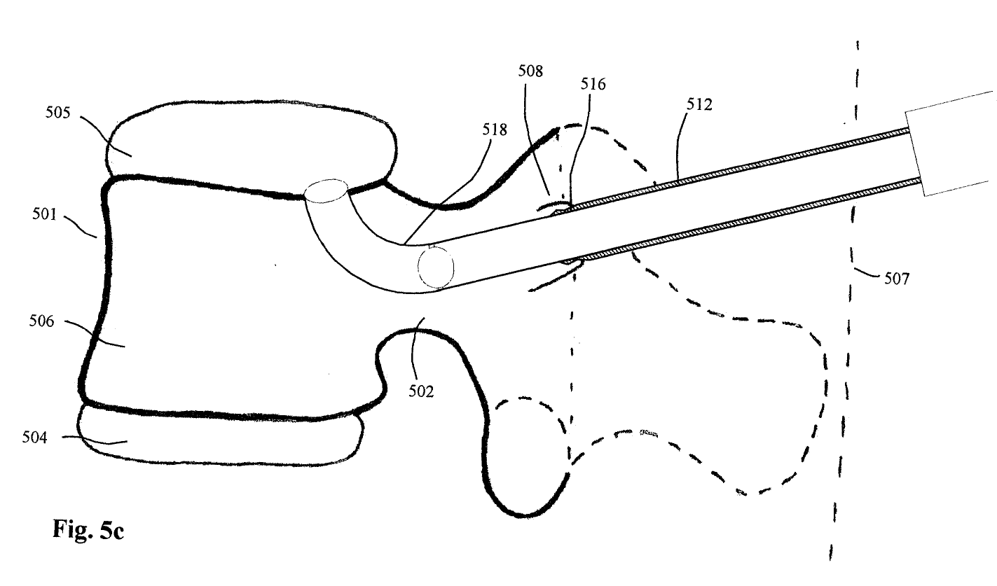

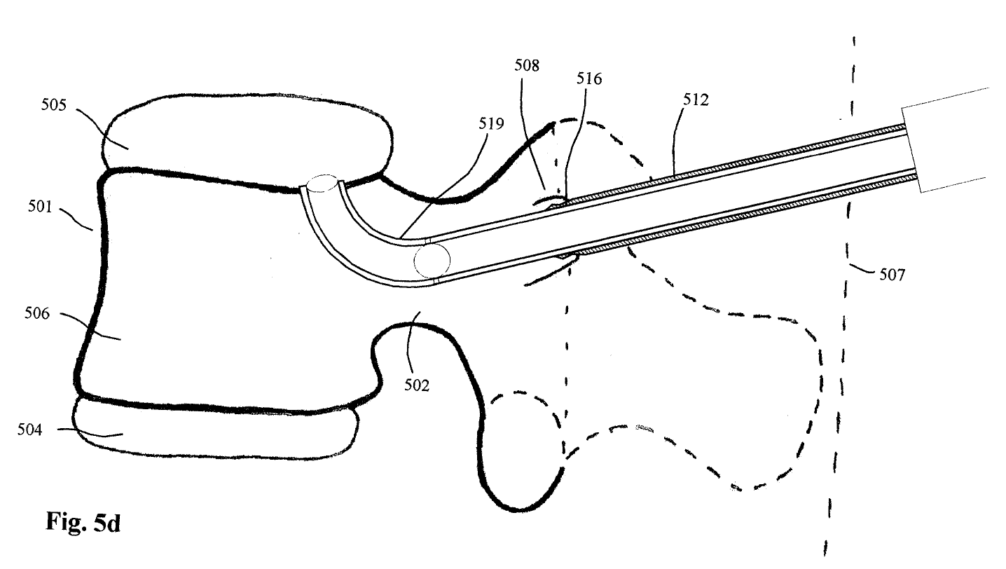

FIG. 5 illustrates one example of the use of the present invention to drill a curved path through the pedicle 502 into the corpus vertebra 506 to reach the superior disc 505. Similar the inferior disc 504 could be reach by turning the instrument 180 degree around its axis. The device of this example comprises an elastic tube 511, a less elastic or stiffer outer tube 512, a less elastic or stiffer inner tube 513, a drill head 514, and a torque wire 515. The drill 514 can be a of any drilling geometry, mechanism or working principle or be a swivel. Not shown in the figure is a suction mechanism drawing the bone shavings out to the proximal side of the device. Further can there be a rinsing mechanism rinsing the tube from the bone shavings.

FIG. 5a. illustrates the step in which the instrument has been pushed by hand force through the patient's skin 507 and soft tissue 509. The most outer less elastic tube 512 comprises a thread 516 at its distal tip, which at this step is screwed into the outer surface 508 of the vertebra 301. The thread is a self cutting thread, which stabilizes the device in respect to the vertebra. An alternative way to anchor the less elastic tube 512 onto the bone would be providing a beveled tip and hammering it into the outer surface 508 of the vertebra 501. The less elastic tube 513 which is inserted in the elastic tube 511 is pushed forward while the torque wire 515 rotates to drive the drill 514. The distal part of the drill 514 located at the tip of the elastic tube 511 has an outer diameter equal to the outer diameter of the elastic tube 511 and the proximal part of the drill 514 located within the elastic tube 511 has an outer diameter equal to the inner diameter of the elastic tube 511. The motor unit located at the proximal side of the instrument to drive the torque wire 515 and consequently the drill 514 is not shown in this figure.

FIG. 5b illustrates the step in which only the elastic tube 511 with rotating drill 514 is further pushed into the vertebra 501. The less elastic or stiff inner tube 513 is not further pushed into the vertebra 501 and halted in the position as shown. Because the more elastic tube 511 is pre-bent in a curved shape (pre-curved), it will bend to its original curved position when not restrained by at least one of the two stiff tubes 512 or 513. The combination of drilling-forward movement and release from tube 513 will result in a curved movement through the vertebra bone 501 until the tip of the drill 514 reaches the disc 505, as illustrated in FIG. 5b.

FIG. 5c illustrates the step in which all inner tubes have been pulled back and the open canal 518, located inside tube 512, remains in place to inject the disc augmenting substance into disc 505. The injecting unit at the proximal end of the instrument is not shown in this figure.

FIG. 5d illustrates the step in which a tube 519 has been inserted to connect to the disc 505. The tube 519 can be an elastic metal or plastic tube.

### 5. Example 3

### Disc Augmentation Utilizing A Balloon

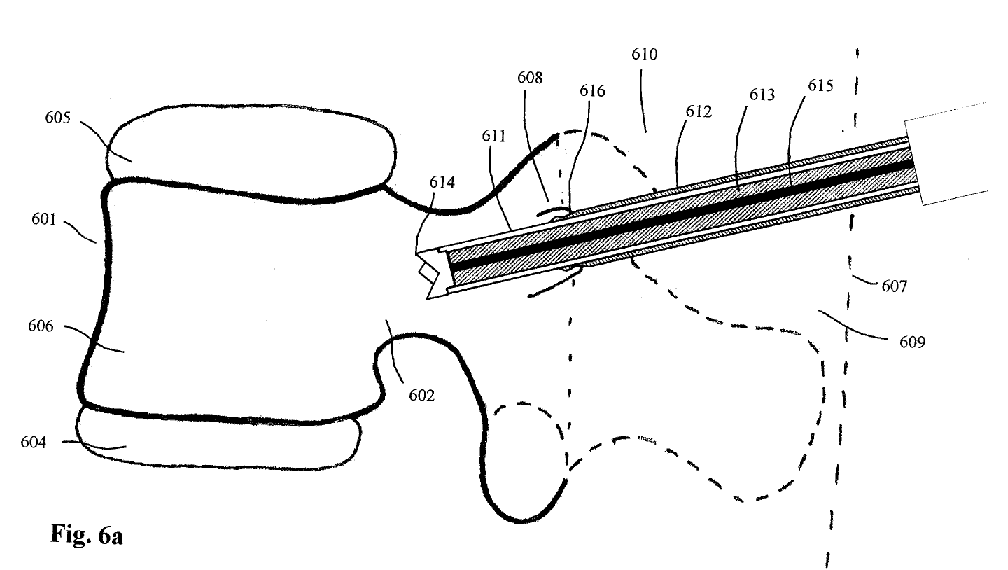

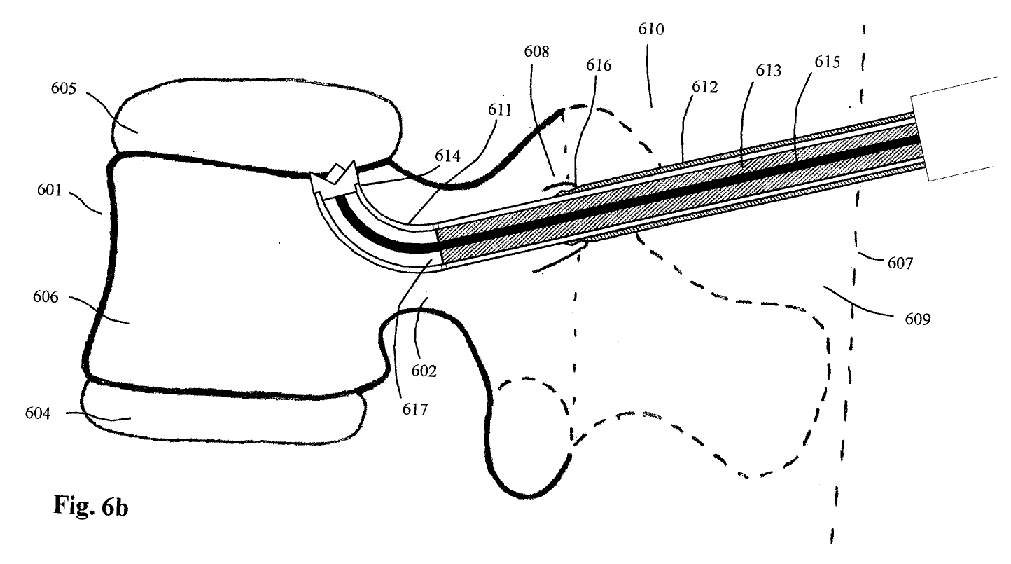

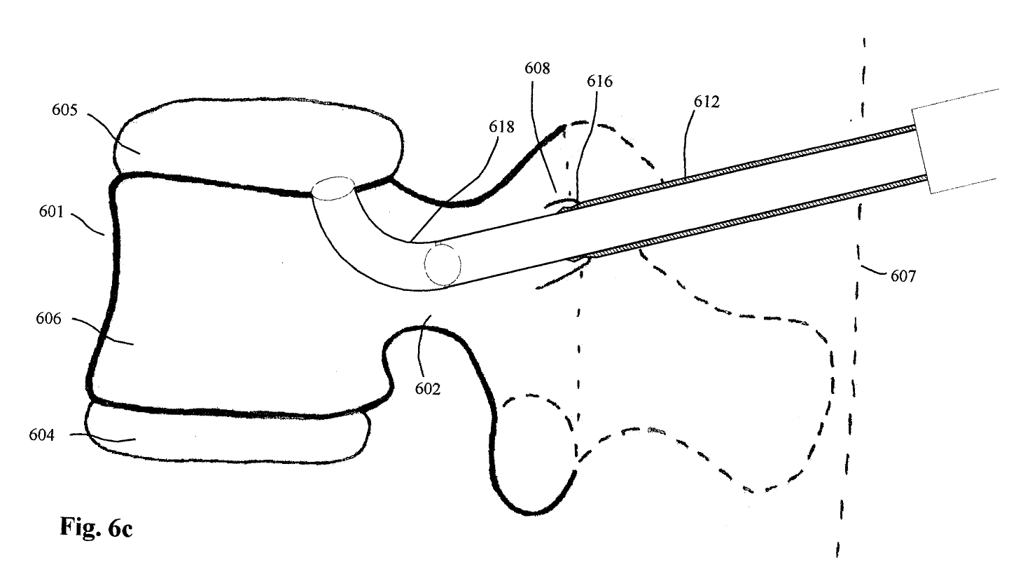

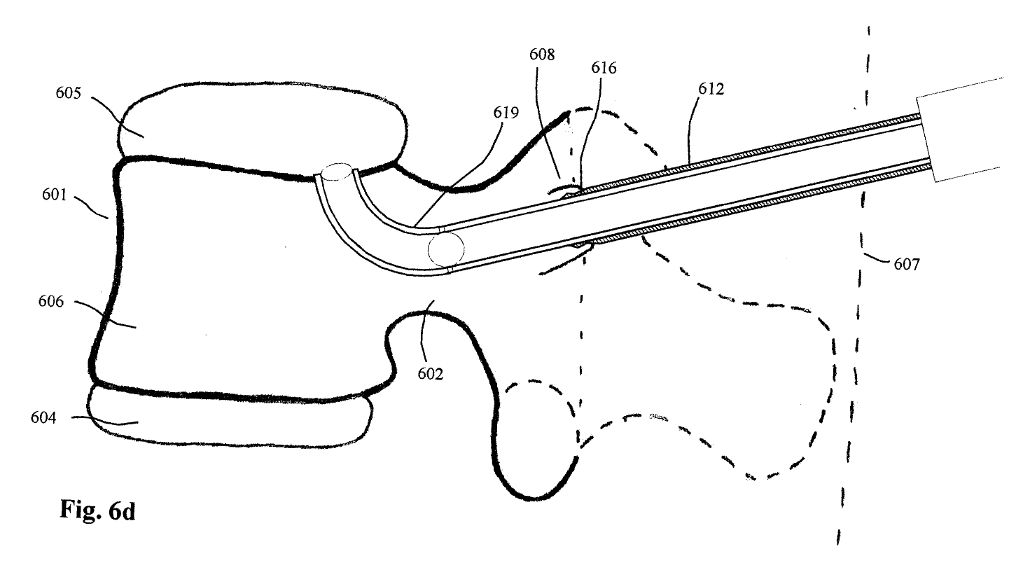

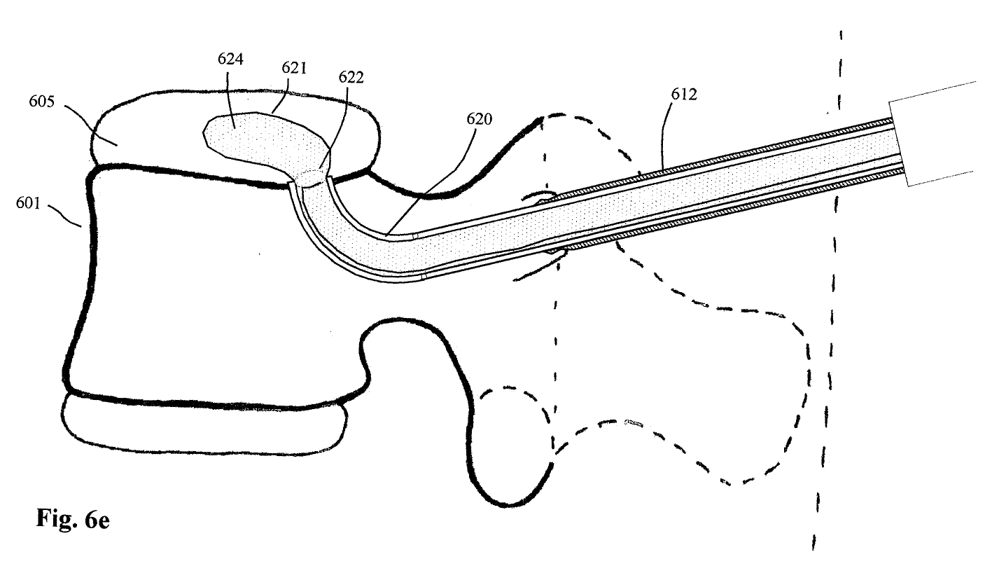

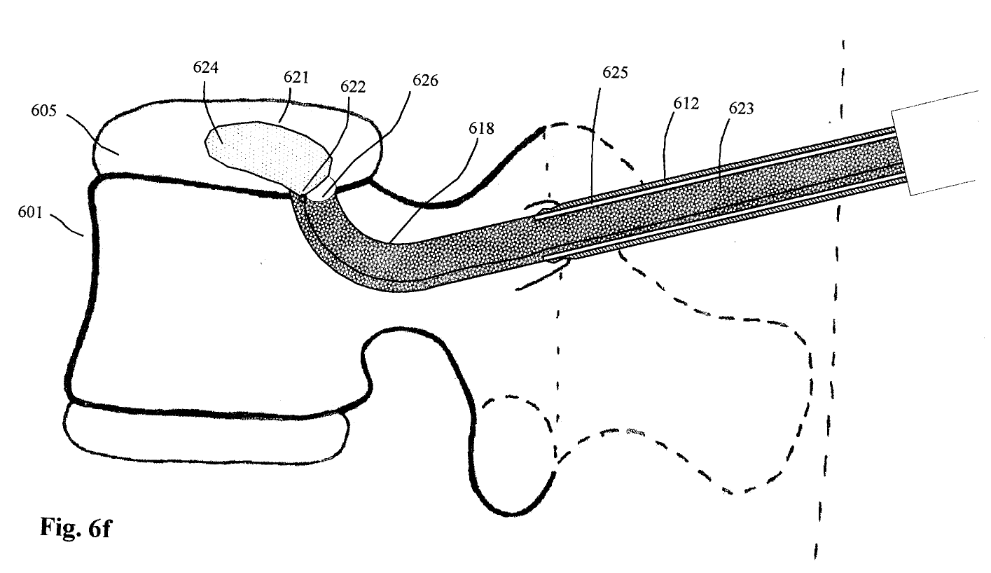

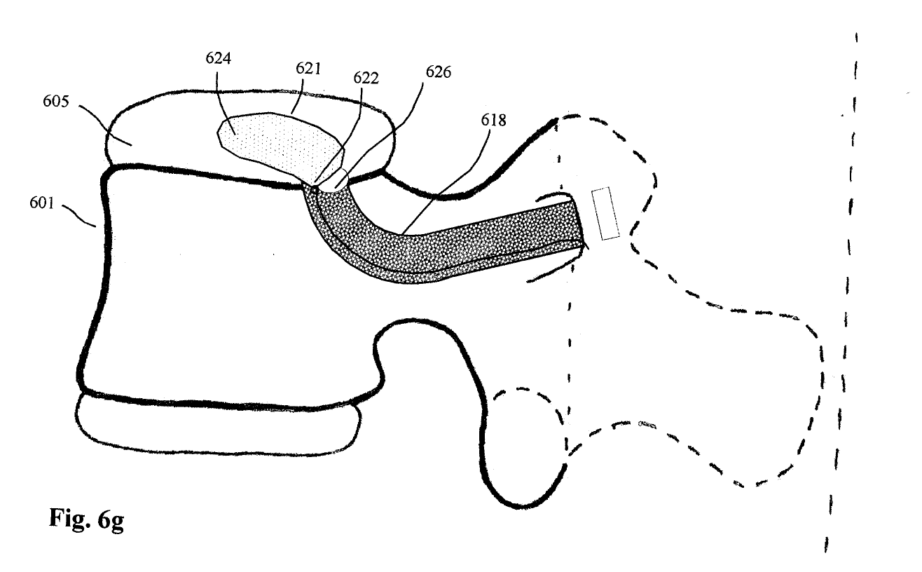

FIG. 6 illustrates one example of the present invention in which the device takes a path through the pedicle 602 into the corpus vertebrae 606 to reach the superior disc 605. Similar the inferior disc 604 could be reached by turning the instrument 180 degree around its axis. The device of this example comprises an elastic tube 611, a less elastic or stiff outer tube 612, a less elastic or stiff inner tube 613, a drill head 614, and a torque wire 615. The drill 614 can be of any drilling geometry, mechanism or working principle or be a swivel. Not shown in the figure is a suction mechanism drawing the bone shavings out to the proximal side of the device. Further, there can be a rinsing mechanism rinsing the tube from the bone shavings.

FIG. 6a. illustrates the step in which the instrument has been pushed by hand force through the patient's skin 607 and soft tissue 609. The outermost less elastic tube 612 comprises a thread 616 at its distal tip, which at this step is screwed into the outer surface 608 of the vertebrae 601. The thread stabilizes the device in respect to the vertebrae. An alternative way to anchor the less elastic tube 612 onto the bone would be a beveled tip which could be hammered into the outer surface 608 of the vertebrae 601. The less elastic tube 613 which is inserted in the elastic tube 611 is pushed forward while the torque wire 615 rotates to drive the drill 614. The distal part of the drill 614 located at the tip of the elastic tube 611 has an outer diameter equal to the outer diameter of the elastic tube 611 and the proximal part of the drill 614 located within the elastic tube 611 has an outer diameter equal to the inner diameter of the elastic tube 611. The motor unit located at the proximal side of the instrument to drive the torque wire 615 and consequently the drill 614 is not shown in this figure.

FIG. 6b illustrates the step in which only the elastic tube 611 with rotating drill 614 is further pushed into the vertebrae 601. The less elastic or stiff inner tube 613 is not further pushed into the vertebrae 601 and is halted in the position as shown. Because the tube 611 is pre-bent in a curved shape (pre-curved), it will bend to its original curved position when not restrained by at least one of the two stiff tubes 612 or 613. The combination of drilling-forward movement and release from tube 613 will result in a curved movement through the vertebrae bone 601 until the tip of the drill 614 will reach the disc 605, as illustrated in FIG. 6b.

FIG. 6c illustrates the step in which all inner tubes have been pulled back and the unobstructed canal 618 remains to inject the disc augmenting substance into the disc 605. The injecting unit at the proximal end of the instrument is not shown in this figure.

FIG. 6d illustrates the step in which a tube 619 has been inserted to connect to the disc 605. The tube 619 can be an elastic metal or plastic tube.

FIG. 6e illustrates one embodiment of the invention in which a balloon 621 is pushed via catheter 620 distally into the disc 605. The balloon, which can be made from silicone rubber, is filled with a disc augmenting substance 624. The advantage of this embodiment is that the disc augmenting substance 624 does not contact the inner tissue of the disc 605, and so avoids any biochemical interactions of the two. A string mechanism 622 can close the balloon 621.

FIG. 6f illustrates the situation in which the balloon 621 has been closed by string sealing mechanism 622, catheter 620 has been pulled out proximally and conventional bone cement 623 has been injected into the canal 618 of the vertebrae 601 to seal the canal.

In FIG. 6g, the outer tube 612 has been removed, along with its contents, and the opening is closed (not shown).

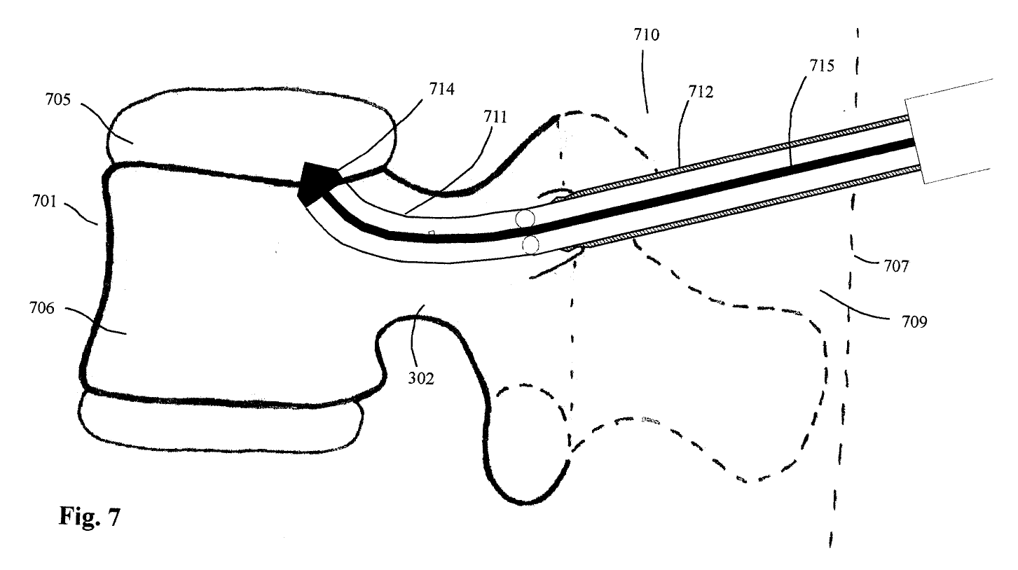

FIG. 7 illustrates another example of the embodiment of the invention to access the disc via a curved path. The instrument 710 is punctured through the skin 707, soft muscle tissue 709 and pedicle 702 of vertebrae 701. Within the corpus vertebrae 706, the elastic tube 711 curves towards the superior disc 705. In a difference from previous examples where the distal part of the drill extends from the elastic tube, in this example the drill 714 does not entirely extend, but is located within the elastic tube 711 but extending its distal tip. The elastic tube 711 is so thin in this example that the elastic effect is caused by the material from which it is made (as in example 1) and from the thin wall thickness of the tube 711. Due to the thin wall thickness it may not be important in this case to bevel the tip of the tube 711. The drill 714 can be made from hardened medical grade stainless steel. The distal tips of tubes 711 and 712 and the drill 714 are circular and beveled with the same angle.

### 6. Example 4

### Disc Augmentation Utilizing Pellets

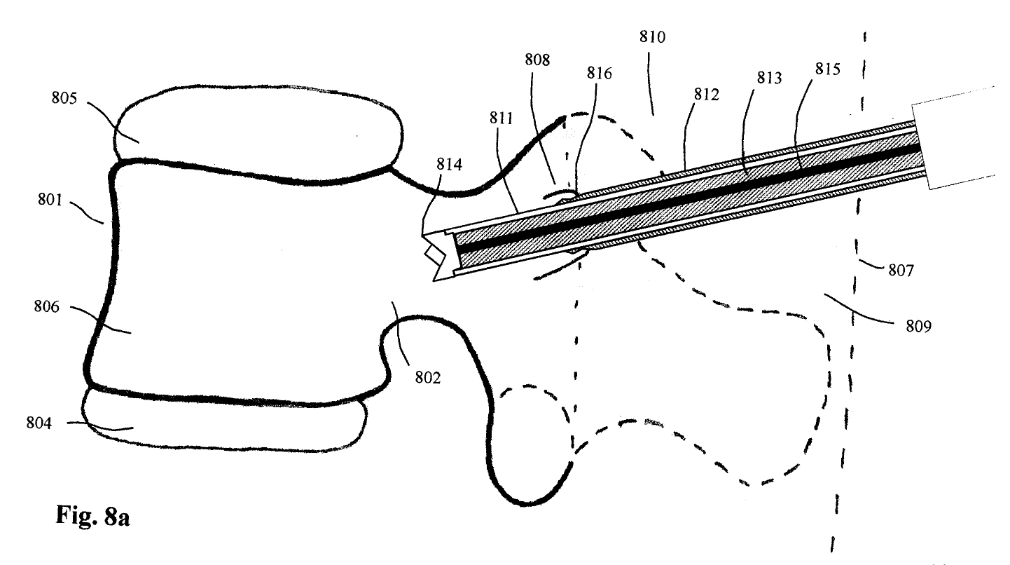

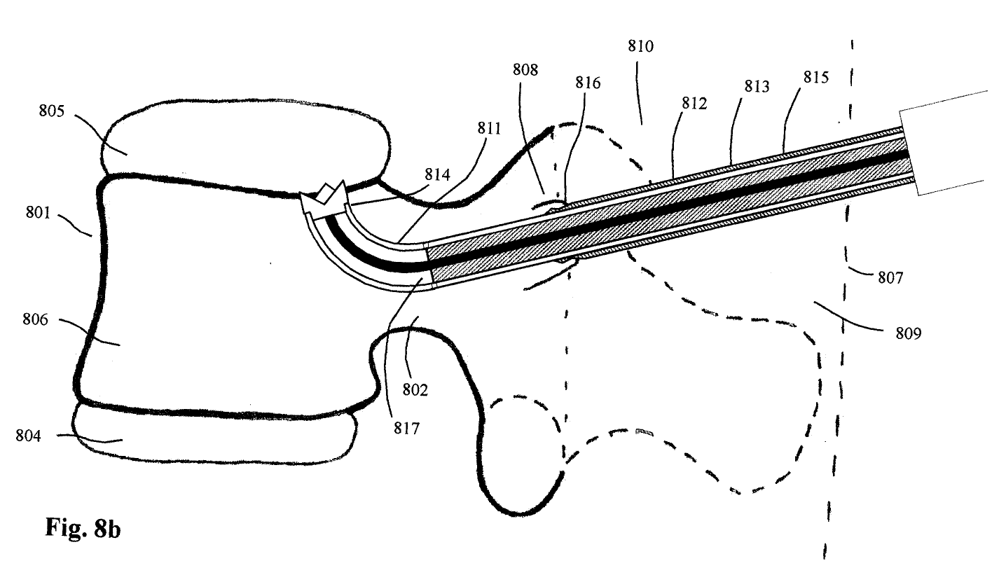

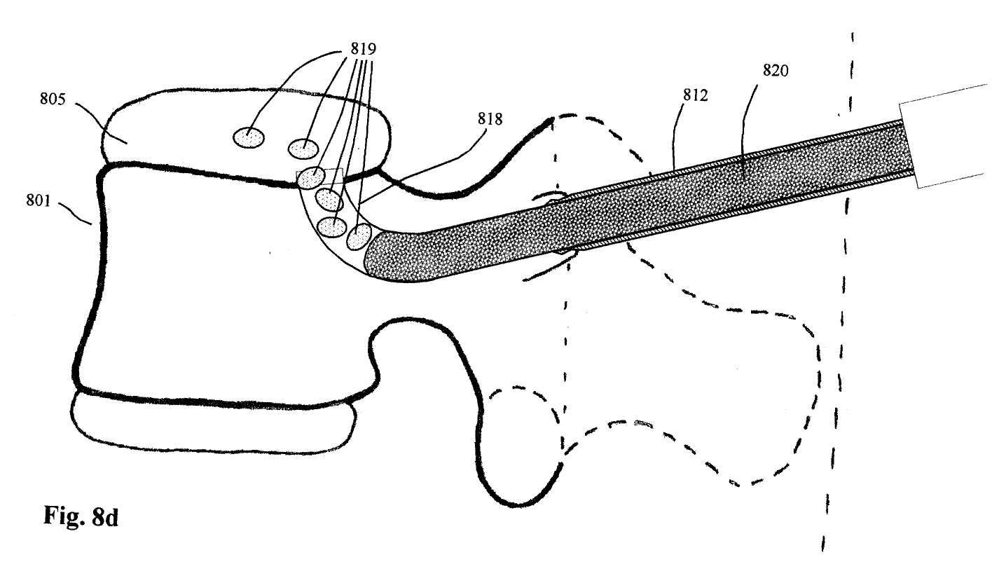

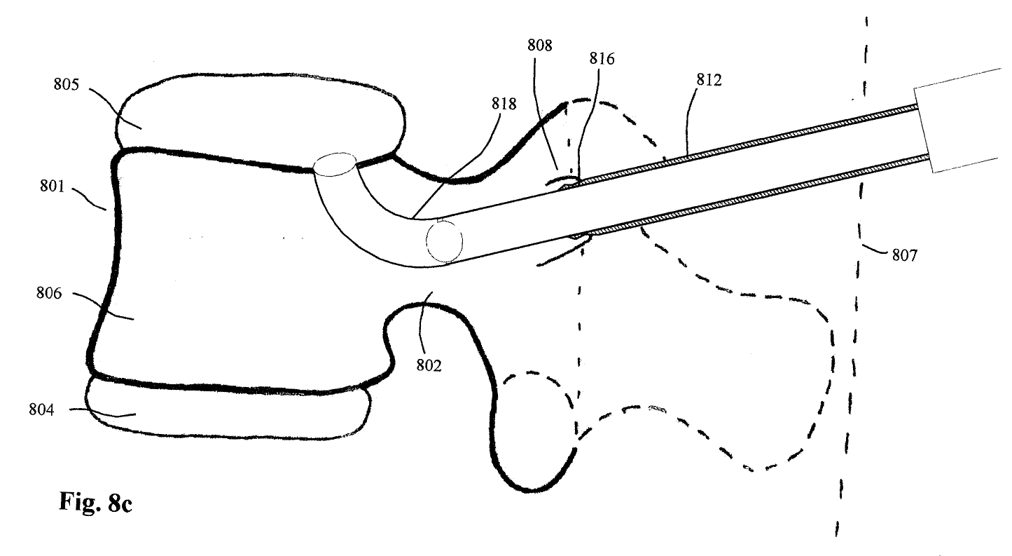

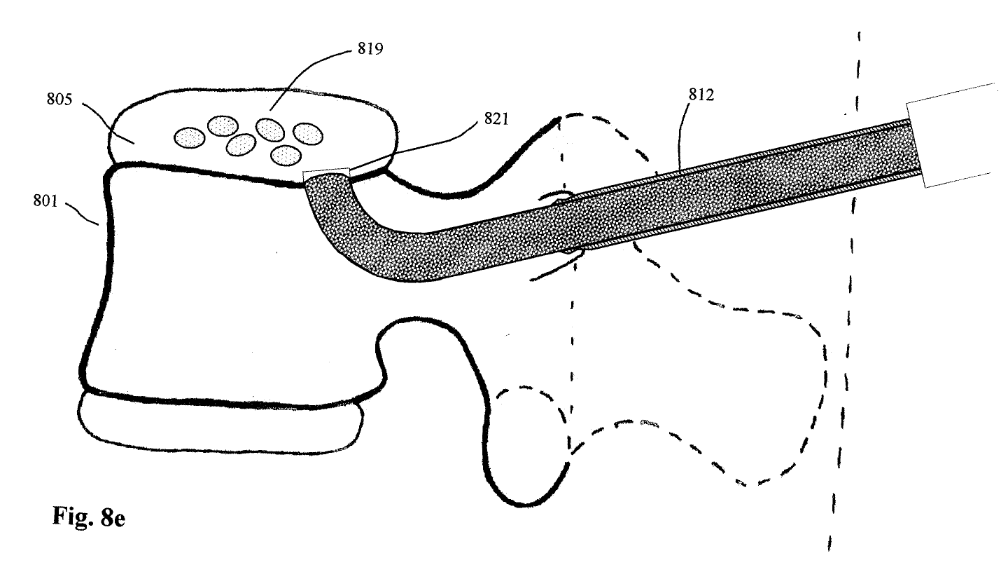

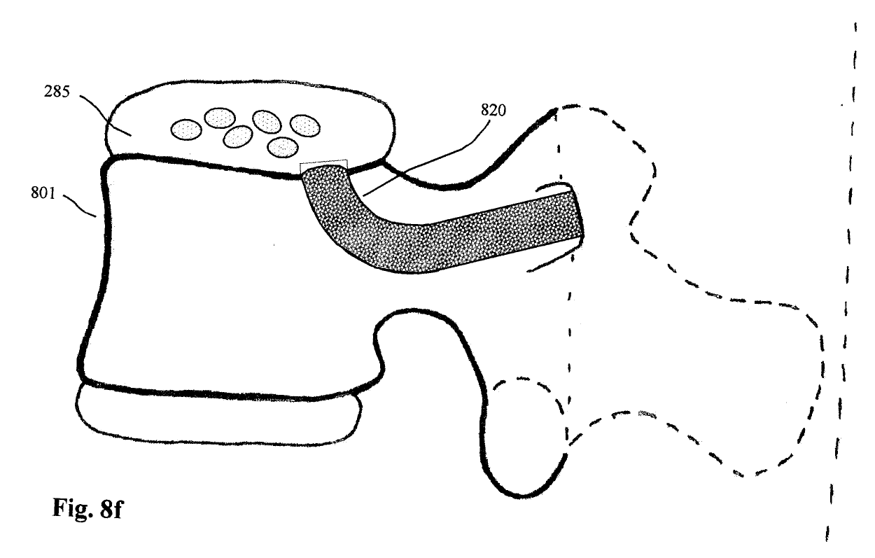

FIG. 8 illustrates one example of the present invention in which the device 810 takes a path through the pedicle 802 into the corpus vertebra 806 to reach the superior disc 805 and to deploy disc augmenting pellets. Similar the inferior disc could be reach by turning the instrument 180 degree around its axis. The device of this example comprises an elastic tube 811, a less elastic or stiff outer tube 812, a less elastic or stiff inner tube 813, a drill head 814, and a torque wire 815. The drill 814 can be of any drilling geometry, mechanism or working principle or be a swivel. Not shown in the figure is a suction mechanism drawing the bone shavings out to the proximal side of the device. Further can there be a rinsing mechanism rinsing the tube from the bone shavings.

FIG. 8a illustrates the step in which the instrument has been pushed by hand force through the patient's skin 807 and soft tissue 809. The most outer less elastic tube 812 comprises a thread 816 at its distal tip, which at this step is screwed into the outer surface 808 of the vertebra 801. The thread is a self cutting thread, which stabilizes the device with respect to the vertebra. An alternative way to anchor the less elastic tube 812 onto the bone would be to provide a beveled tip and hammer it into the outer surface 808 of the vertebra 801. The less elastic tube 813 which is inserted in the elastic tube 811 is pushed forward while the torque wire 815 rotates to drive the drill 814. The distal part of the drill 814 located at the tip of the elastic tube 811 has an outer diameter equal to the outer diameter of the elastic tube 811 and the proximal part of the drill 814 located within the elastic tube 811 has an outer diameter equal to the inner diameter of the elastic tube 811. The motor unit located at the proximal side of the instrument to drive the torque wire 815 and consequently the drill 814 is not shown in this figure.

FIG. 8b illustrates the step in which only the elastic tube 811 with rotating drill 814 is further pushed into the vertebra 801. The less elastic or stiff inner tube 813 is not further pushed into the vertebra 801, and is halted in the position as shown. Because the tube 811 is pre-bent in a curved shape (pre-curved), it will bend to its original curved position when not restrained by at least one of the two stiff tubes 812 or 813. The combination of drilling-forward movement and release from tube 813 will result in a curved movement through the vertebra bone 801 until the tip of the drill 814 will reach the disc 805, as illustrated in FIG. 8b.

FIG. 8c illustrates the step in which all inner tubes have been pulled back and the open canal 818 to the disc 805 remains.

FIG. 8d illustrates the step in which tablets 819 (6 tablets in this example) are pushed through the canal 818 into the disc 805. In this example the tablets 819 are pushed by bone cement 820. Alternatively, the pushing could be provided by an elastic rod such as a rubber or metal rod to push the tablets forward into the disc, or by other means producing the same effect.

FIG. 8e illustrates the step in which the pushing bone cement reaches the endplate of the vertebra and all tablets 819 are deployed into the disc, filling the volume of the nucleus pulposus and thus adjusting disc height and disc elasticity.

FIG. 8f illustrates the step in which all instrumentation has been removed. All tablets are deployed in the nucleus pulposus and the canal in the vertebra is sealed by bone cement.

A tablet can be a small and rounded mechanical device, pill, capsule, pellet, caplet or the like. It can be made from an elastic rubber type material. A tablet can be in its simplest form a rounded piece, made of silicon or polyurethane rubber. A tablet can also be a small plastic, metal or rubber capsule or rubber balloon type device filled with a substance like a hydrogel or with a colloidal suspension or a powder. Typically the tablets are of size 1 to 10 millimeter, preferably below 5 millimeter.

Just like the balloon or balloons in the previous example above, the tablets can be anchored by strings into the channel sealing bone cement. The strings can be made in both examples from biocompatible materials such as plastics or metal.

The mechanism of all shown examples above may be used to inject radiographic contrast media or may be used to insert any surgical instrumentation for disc surgery. The curved path may also be used to remove disc tissue. In some instances the curved path may be sealed with other means than bone cement, for example with a mechanical stopper (e.g. rubber plug, block, cover, screw), or it may not be permanently sealed at all, and left with a removable closure for the purpose of future access to the disc.

The advantage of the mechanisms as discussed in the examples is that the outer layer of the disc, the annulus fibrosus, is left untouched and intact. Thus, with the blockage of the entry pathway by bone cement or other filler, the contents of the annulus are held inside the annulus, and may be replaced or maintained by natural processes. On the other hand the methods disclosed here require rupture of an endplate of the disc-adjacent vertebra which is of importance for the disc's stability and its local metabolism. However, in a typical practical scenario, a lumbar disc on an endplate will lose less than 5% of its active area. Since the disc has two adjacent endplates the area opening is likely be below 2.5% of the disc area adjacent to the vertebrae. This is probably more acceptable than a rupture in the annulus fibrosus, which can build a leak and become a seed for further rupture.

### 7. System

The system for creating a curved disc access path comprises a telescopic tube assembly of the kinds described in the above examples, and further comprises a motor unit providing the rotation for the drill, and a system-positioning unit. The system-positioning unit can be in its simplest form a hand piece which operates like a conventional electric powered drill, where pulling a lever results in the rotation of the drill. A more sophisticated system could be attached to the patient positioning table of the radiographic imaging system, allowing a more stereotactic procedure. The tube or needle assembly is preferably disposable and is attached to the system-positioning unit for the duration of the procedure. The motor unit can be powered electrically, pneumatically, hydraulically, by mechanical spring loaded mechanism, or by hand winding. The motor unit can be an integral part of the system-positioning system or be attached to the needle assembly only when needed. The drilling unit can be a mechanical drill as described in the above examples or a laser ablation system.

The system may further comprise a suction unit for drawing the bone shavings out of the drilling area and a rinsing unit rinsing the tube from the bone shavings. For the disc augmenting procedure the patient can be positioned in the supine, side, lateral, seated, spine bend forward and prone position. The procedure may be performed under general or localized anesthesia.

### 8. Dimensions And Materials

The angle by which the pre-bend tubes displaces from a straight line (which is given by the rigid straight tube) must be greater than one degree and up to 90 degrees. The radius of the pre-bend tube can be between 5 millimeter (mm) and 1000 millimeter. The rotation speed can be any conventional speed, and typically will be between 1 rotation per minute and 10,000 rotation per minute (RPM).

The less elastic tube in the above examples can be made from any medical grade material, including stainless steel, plastic, carbon fiber or any combination thereof. In the event that the instrument is used under magnetic imaging (MR) guidance, the material is based on non-magnetically-susceptible materials, for example titanium, which are preferably of ASTM Grade 9, and at least harder than an alloy according to ASTM Grade 5 or ISO 3.765 or 3.7165. Typically, the wall-thickness of an outer tube is in the range of from about 0.01 millimeters to about 1.5 millimeters. The outer diameter of the elastic tube can be between 0.5 millimeter and 5 millimeter, and the outer diameter of the entire inserted instrument between 0.5 millimeter and 10 millimeter, preferably between 3 millimeter and 6 millimeter.

The elastic tube in above examples can be made from very durable elastic steels, titanium-vanadium-alloys, plastic, carbon fibre, nickel-titanium (NiTi), or super-elastic nickel-titanium (NiTi), also known as Nitinol. The elasticity of NiTi is in the range of approximately 83 gigapascal (GP) a (austenite) to approx. 28 to 41 GPa (martensite). An example of another good medical material is stainless steel 316L with modulus of elasticity of around 193 GPa in tension and about 77 GPa in torsion. Suitable materials include nickel-chrome-alloy such as ASTM F563-78 comprising 15-25% nickel, 18-22% chromium, up to 4% titanium, up to 4% molybdenum and, up to 6% iron. This material can be purchased from Institute Straumann in 4437 Waldenburg, Switzerland under the trademark “SYNTACOBEN”. Similar material can be purchased from General Resorts SA in 2501 Bienne, Switzerland under the trademark “NIVAFLEX”. A further useful material is “DURATHERM 600” which is a Co—Ni—Cr—Mo—W alloy. Typically, the wall-thickness of the pre-bend tube is in the range of from about 0.01 millimeters to about 1.0 millimeters. In general all materials which are used for mechanical springs are candidates for the inner tube. The torque-wire is preferably made of flexible metal material, such as stainless steel.

### 9. Radiographic Guidance

The disc augmenting procedure is preferably performed under radiographic image guidance. In one embodiment of the present invention, the device is guided by x-ray fluoroscopy, computer tomography, magnetic resonance imaging, ultrasound, visual, positron emission tomography, single photon emission computed tomography, or any combination thereof. In some instances it is beneficial to deploy and leave a marker in the disc for later radiographic control. Such a marker can be a little pellet made from any biocompatible and radiographically visible material, such as stainless steel, plastic or titanium alloy.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

What is claimed is:

1. A device for creating a transpedicular delivery canal into a spinal disc for the treatment of degenerative disc disease, said device comprising:
an elastic pre-curved tube,
at least one less elastic tube, and
a drill which is positioned at the distal tip of the device and which is driven by a motor unit,
wherein said less elastic tube is capable of bending said pre-curved elastic tube so that the pre-curved tube is straight;
and wherein said pre-curved elastic tube slides on the outside of said less elastic tube.

2. A device according to claim 1 wherein the material of said less elastic tube is selected from the group consisting of nickel-titanium; super-elastic nickel-titanium; nickel-chrome-alloy; nickel-chrome-alloy ASTM F563-78 comprising 15-25% nickel, 18-22% chromium, up to 4% titanium, up to 4% molybdenum and, up to 6% iron; Co—Ni—Cr—Mo—W alloy, and any combination thereof.

3. A device according to claim 1 wherein said drill is connected to said motor via a torque wire.

4. A device according to claim 1 further comprising one or both of a suction unit and a rinsing unit.

5. A device according to claim 1, further comprising a deployable treatment unit.

6. A device according to claim 6 in which the treatment unit comprises a disc augmenting gel.

7. A device according to claim 6 in which the treatment unit comprises a radiographic contrast medium.

8. A device according to claim 6 in which the treatment unit comprises at least one balloon

9. A device according to claim 6 in which the treatment unit comprises one or more pellets.

10. A method for creating a delivery canal into a spinal disc, said method comprising:
providing an elastic pre-curved tube;
providing at least one less elastic tube which is capable of bending said pre-curved elastic tube so that it is straight;
and providing a drill which is positioned on the distal tip of the device and which is connected via a torque wire to a motor unit;
wherein said at least one less elastic tube is capable of bending said pre-curved elastic tube so that it is straight;
and wherein said pre-curved elastic tube slides on the outside of said less elastic tube;
wherein said delivery canal is created by drilling into the pedicle of a vertebra sufficiently to provide access through the vertebra to the spinal disc, with said pre-curved tube riding on said less elastic tube;
and at a selected point, passing said pre-curved tube over said less elastic tube so that the precurved tube is directed towards a surface of said spinal disc; advancing said precurved tube into said disc; introducing treatment materials into said disc; withdrawing said precurved tube and said less elastic tube from the vertebra; and filling the opening created by said drill with a cement.

11. The method of claim 10 wherein said treatment materials comprise one or more of a balloon, a pellet, a gel, and a therapeutic substance.

12. The method of claim 10 wherein the material of said less elastic tube is selected from the group consisting of nickel-titanium; super-elastic nickel-titanium; nickel-chrome-alloy; nickel-chrome-alloy ASTM F563-78 comprising 15-25% nickel, 18-22% chromium, up to 4% titanium, up to 4% molybdenum and, up to 6% iron; Co—Ni—Cr—Mo—W alloy, and any combination thereof.

13. The method of claim 10 wherein said drill is connected to said motor via a torque wire.

14. The method of claim 10 further comprising one or more of a suction unit, a rinsing unit, and a treatment unit.

15. The method of claim 15 in which the treatment unit comprises one or more of a disc augmenting gel, a radiographic contrast medium, at least one balloon, and one or more pellets.

16. A system to treat the spinal disc, the system comprising a mechanical component and a method of use,
wherein said mechanical component comprises:
an elastic pre-curved tube;
at least one less elastic tube which is capable of bending said pre-curved elastic tube so that the pre-curved tube is straight and slides on said less elastic tube;
and a drill which is positioned at the distal tip of the device and which is driven by a motor unit, and a source of a disc-stabilizing material;
and wherein said pre-curved elastic tube slides on the outside of said less elastic tube;
and wherein said method of use comprises the steps of using said mechanical component to create a trans-pedicular pathway passing from the pedicle of a vertebra into an adjacent spinal disc; creating an opening into said vertebra; injecting materials to enhance the function of said spinal disc; withdrawing said mechanical components from said vertebra; and sealing the openings in said disc and in at least adjacent areas of the created pathway through said vertebra with a cement.

17. The system of claim 16 wherein the injected materials form a gel in said disc.

18. The system of claim 16 wherein said injected materials enhance disc function by increasing the volume of material inside said disc.

19. The system of claim 18 wherein said injected materials contain one or more of a gelling material, one or more pellets, and a balloon inflated with a fluid while inside said disc.

20. The system of claim 16, further comprising a responsive disc augmentation system, which measures the pressure in the spinal disc and releases disc augmenting substance into the disc when the pressure drops under a critical value, and can be set for autonomous or manual augmentation release.
